(19) United States
(12) Patent Application Publication
(10) Pub. No.: US 2007/0093548 A1
(43) Pub. Date: Apr. 26, 2007

(54) Use of progesterone receptor modulators

(75) Inventors: Jason Michael Diffendal, Pottstown, PA (US); Cuijian Yang, Collegeville, PA (US); Richard Eric Mewshaw, Pottstown, PA (US)

Correspondence Address: HOWSON AND HOWSON;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 11/546,785

(22) Filed: Oct. 12, 2006

Related U.S. Application Data
(60) Provisional application No. 60/730,094, filed on Oct. 25, 2005.

(51) Int. Cl. A61K 31/343; C07D 307/91

(52) U.S. Cl. 514/468; 549/460

(57) ABSTRACT

A progesterone receptor modulator of the structure 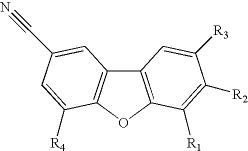 is provided. Use of compositions containing this compound for contraception, hormone replacement therapy, treating hormone-dependent disease, synchronizing estrus, treating dysmenorrhea, treating dysfunctional uterine bleeding, inducing amenorrhea, or treating symptoms of premenstrual syndrome and premenstrual dysphoric disorder in a mammal are described.

### CROSS-REFERENCE TO RELATED APPLICATIONS

This application claims the benefit of priority under 35 USC §119(e) of U.S. Provisional Patent Application No. 60/730,094, filed Oct. 25, 2005.

### BACKGROUND OF THE INVENTION

Progesterone receptor (PR) agonists and antagonists, also termed PR modulators, have been described for use in contraception and a variety of other indications.

Intracellular receptors (IR) form a class of structurally related gene regulators known as “ligand dependent transcription factors”. Once a ligand is present in the fluid surrounding a cell, it passes through the membrane via passive diffusion, and binds to the IR to create a receptor/ligand complex. This complex binds to specific gene promoters present in the cell's DNA. Once bound to the DNA the complex modulates the production of mRNA and the protein encoded by that gene.

A compound that binds to an IR and mimics the action of the natural hormone is termed an agonist, whilst a compound which inhibits the effect of the hormone is an antagonist.

The steroid receptor family is a subset of the IR family, including progesterone receptor (PR), estrogen receptor (ER), androgen receptor (AR), glucocorticoid receptor (GR), and mineralocorticoid receptor (MR). The natural hormone, or ligand, for the PR is the steroid progesterone, but synthetic compounds, such as medroxyprogesterone acetate or levonorgestrel, have been made which also serve as PR ligands.

PR agonists (natural and synthetic) are known to play an important role in the health of women. PR agonists are used in birth control formulations, either along or in the presence of an ER agonist. ER agonists are used to treat the symptoms of menopause, but have been associated with a proliferative effect on the uterus which can lead to an increased risk of uterine cancers.

PR antagonists have also been described as being useful for the treatment of hormone dependent breast cancers, hormone dependent prostate cancer, and non-malignant chronic conditions such as uterine fibroids, endometriosis, and in hormone replacement therapy. Such a use may be alone or in combination with a partial ER antagonist, such as tamoxifen. Additionally, PR antagonists may be used in contraception. In this context they may be administered alone, in combination with a PR agonist, or in combination with a partial ER antagonist such as tamoxifen.

What are needed are novel PR modulators.

### SUMMARY OF THE INVENTION

In one aspect, the present invention provides novel PR modulators and uses thereof in hormone replacement therapy, for synchronizing estrus, and for treating contraception, hormone neoplastic disease, dysmenorrhea, dysfunctional uterine bleeding, the symptoms of premenstrual syndrome and premenstrual dysphoric disorder, and for inducing amenorrhea.

In yet a further aspect, the present invention provides kits containing the compounds of the invention.

Other aspects and advantages of the present invention are described further in the following detailed description of the preferred embodiments.

### DETAILED DESCRIPTION OF THE INVENTION

The invention provides methods and products useful for contraception, hormone replacement therapy, synchronizing estrus, treating dysmenorrhea, treating dysfunctional uterine bleeding, inducing amenorrhea, cycle-related symptoms, or treating symptoms of premenstrual syndrome and premenstrual dysphoric disorder. The invention involves administering to a female mammal in need thereof a pharmaceutically effective amount of a compound having the structure of formula I, or a pharmaceutically acceptable salt thereof:

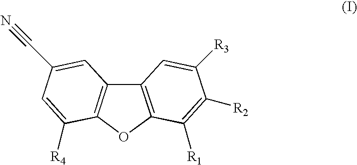

wherein R₁ is selected from the group consisting of hydrogen, halogen, alkyl, substituted alkyl, aryl, substituted aryl, heteroaryl, and substituted heteroaryl;

R₂ is selected from the group consisting of hydroxyl, alkoxy, and substituted alkoxy;

R₃ is selected from the group consisting of hydrogen and halogen; and

R₄ is selected from the group consisting of hydrogen and halogen.

In one embodiment, in the compound of formula I, R₁ is selected from the group consisting of hydrogen, halogen, wherein the halogen is bromine, alkyl, wherein the alkyl is C₁-C₆ alkyl, and substituted aryl, wherein the aryl is phenyl.

In another embodiment, in the compound of formula I, R₂ is selected from hydroxy or methoxy.

In still another embodiment, in the compound of formula I, R₃ is selected from hydrogen, fluorine or bromine.

In yet another embodiment, R₄ is hydrogen or fluorine.

In a further embodiment, R₁ is a C₃-C₄ alkyl; R₂ is hydroxy; R₃ is H; and R₄ is H.

In yet a further embodiment, R₁ is a C₃-C₄ alkyl; R₂ is methoxy; R₃ is H; and R₄ is H.

The compounds utilized according to one embodiment of the present invention can contain one or more asymmetric centers and can thus give rise to optical isomers and diastereomers. For example, in one embodiment, R₁ is an alkyl having a chiral center. In one embodiment, the chiral center is in the R-configuration. In another embodiment, the chiral center is in the S-configuration. While shown without respect to stereochemistry, the compounds can include optical isomers and diastereomers; racemic and resolved enantiomerically pure R and S stereoisomers; other mixtures of the R and S stereoisomers; and pharmaceutically acceptable salts thereof.

The term “alkyl” is used herein to refer to both straight- and branched-chain saturated aliphatic hydrocarbon groups having about 1 to about 8 carbon atoms, and desirably about 1 to about 6 carbon atoms (i.e., C₁, C₂, C₃, C₄, C₅ or C₆). The term “lower alkyl” generally refers to alkyls having 1, 2, 3, or 4 carbons.

The term “alkenyl” is used herein to refer to both straight- and branched-chain alkyl groups having one or more carbon-carbon double bonds and containing about 3 to about 8 carbon atoms. Desirably, the term alkenyl refers to an alkyl group having 1 or 2 carbon-carbon double bonds and having 3 to about 6 carbon atoms.

The term “alkynyl” group is used herein to refer to both straight- and branched-chain alkyl groups having one or more carbon-carbon triple bonds and having 3 to about 8 carbon atoms. Desirably, the term alkynyl refers to an alkyl group having 1 or 2 carbon-carbon triple bonds and having 3 to about 6 carbon atoms.

The terms “substituted alkyl”, “substituted alkenyl”, and “substituted alkynyl” refer to alkyl, alkenyl, and alkynyl groups, respectively, having one or more substituents including, without limitation, halogen, CN, OH, NO₂, amino, aryl, heterocyclic groups, aryl, alkoxy, aryloxy, alkyloxy, alkylcarbonyl, alkylcarboxy, amino, and arylthio.

The term “acyl” as used herein refers to a carbonyl substituent, i.e., a C(O)(R) group where R is a straight- or branched-chain saturated aliphatic hydrocarbon group including, without limitation, alkyl, alkenyl, and alkynyl groups. Desirably, the R groups have 1 to about 8 carbon atoms, and more desirably 1 to about 6 carbon atoms. The term “substituted acyl” refers to an acyl group which is substituted with 1 or more groups including halogen, CN, OH, and NO₂.

The term “aryl” as used herein refers to an aromatic system which can include a single ring or multiple aromatic rings fused or linked together where at least one part of the fused or linked rings forms the conjugated aromatic system. The aryl groups include, but are not limited to, phenyl, naphthyl, biphenyl, anthryl, tetrahydronaphthyl, phenanthryl, indene, benzonaphthyl, fluorenyl, and carbazolyl.

The term “substituted aryl” refers to an aryl group which is substituted with one or more substituents including halogen, CN, OH, NO₂, amino, alkyl, cycloalkyl, alkenyl, alkynyl, alkoxy, aryloxy, alkyloxy, alkylcarbonyl, alkylcarboxy, alkylamino, and arylthio. Desirably, a substituted aryl group is substituted with 1 to about 4 substituents.

The term “heterocyclic” as used herein refers to a stable 4- to 7-membered monocyclic or multicyclic heterocyclic ring which is saturated, partially unsaturated, or wholly unsaturated. The heterocyclic ring has in its backbone carbon atoms and one or more heteroatoms including nitrogen, oxygen, and sulfur atoms. Desirably, the heterocyclic ring has about 1 to about 4 heteroatoms in the backbone of the ring. When the heterocyclic ring contains nitrogen or sulfur atoms in the backbone of the ring, the nitrogen or sulfur atoms can be oxidized. The term “heterocyclic” also refers to multicyclic rings in which a heterocyclic ring is fused to an aryl ring. The heterocyclic ring can be attached to the aryl ring through a heteroatom or carbon atom provided the resultant heterocyclic ring structure is chemically stable.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Oxygen-containing rings include, but are not limited to, furyl, tetrahydrofuranyl, pyranyl, pyronyl, and dioxinyl rings. Nitrogen-containing rings include, without limitation, pyrrolyl, pyrazolyl, imidazolyl, triazolyl, pyridyl, piperidinyl, 2-oxopiperidinyl, pyridazinyl, pyrimidinyl, pyrazinyl, piperazinyl, azepinyl, triazinyl, pyrrolidinyl, and azepinyl rings. Sulfur-containing rings include, without limitation, thienyl and dithiolyl rings. Mixed heteroatom containing rings include, but are not limited to, oxathiolyl, oxazolyl, thiazolyl, oxadiazolyl, oxatriazolyl, dioxazolyl, oxathiazolyl, oxathiolyl, oxazinyl, oxathiazinyl, morpholinyl, thiamorpholinyl, thiamorpholinyl sulfoxide, oxepinyl, thiepinyl, and diazepinyl rings. Fused heteroatom-containing rings include, but are not limited to, benzofuranyl, thionapthene, indolyl, benazazolyl, purindinyl, pyranopyrrolyl, isoindazolyl, indoxazinyl, benzoxazolyl, anthranilyl, benzopyranyl, quinolinyl, isoquinolinyl, benzodiazonyl, napthylridinyl, benzothienyl, pyridopyridinyl, benzoxazinyl, xanthenyl, acridinyl, and purinyl rings.

The term “substituted heterocyclic” as used herein refers to a heterocyclic group having one or more substituents including halogen, CN, OH, NO₂, amino, alkyl, cycloalkyl, alkenyl, alkynyl, alkoxy, aryloxy, alkyloxy, alkylcarbonyl, alkylcarboxy, alkylamino, and arylthio. Desirably, a substituted heterocyclic group has 1 to 4 substituents.

The term “arylthio” as used herein refers to the S(aryl) group, where the point of attachment is through the sulfur-atom and the aryl group can be optionally substituted as defined above. The term “alkoxy” as used herein refers to the O(alkyl) group, where the point of attachment is through the oxygen-atom and the alkyl group is optionally substituted as defined above. The term “aryloxy” as used herein refers to the O(aryl) group, where the point of attachment is through the oxygen-atom and the aryl group is optionally substituted as defined above.

The term “alkylcarbonyl” as used herein refers to the C(O)(alkyl) group, where the point of attachment is through the carbon-atom of the carbonyl moiety and the alkyl group is optionally substituted as defined above.

The term “alkylcarboxy” as used herein refers to the C(O)O(alkyl) group, where the point of attachment is through the carbon-atom of the carboxy moiety and the alkyl group is optionally substituted as defined above.

The term “aminoalkyl” as used herein refers to both secondary and tertiary amines where the point of attachment is through the nitrogen-atom and the alkyl groups are optionally substituted as defined above. The alkyl groups can be the same or different.

The term “halogen” as used herein refers to Cl, Br, F, or I groups.

The compounds useful in the present invention may encompass tautomeric forms of the structures provided herein characterized by the bioactivity of the drawn structures. Further, the compounds useful in the present invention may be used in the form of salts derived from pharmaceutically or physiologically acceptable bases, alkali metals and alkaline earth metals.

Pharmaceutically acceptable salts may be formed from inorganic bases, desirably alkali metal salts, for example, sodium, lithium, or potassium, and organic bases, such as ammonium, mono-, di-, and trimethylammonium, mono-, di- and triethylammonium, mono-, di- and tripropylammonium (iso and normal), ethyldimethylammonium, benzyldimethylammonium, cyclohexylammonium, benzylammonium, dibenzylammonium, piperidinium, morpholinium, pyrrolidinium, piperazinium, 1-methylpiperidinium, 4-ethylmorpholinium, 1-iso-propylpyrrolidinium, 1,4-dimethylpiperazinium, 1-n-butyl piperidinium, 2-methylpiperidinium, 1-ethyl-2-methylpiperidinium, mono-, di- and triethanol-ammonium, ethyl diethanolammonium, n-butylmonoethanolammonium, tris(hydroxymethyl)methylammonium, phenylmonoethanolammonium, and the like.

Physiologically acceptable alkali. salts and alkaline earth metal salts can include, without limitation, sodium, potassium, calcium and magnesium salts. These salts, as well as other compounds useful in the invention can be in the form of esters, carbamates and other conventional “pro-drug” forms, which, when administered in such form, convert to the active moiety in vivo. Other conventional “pro-drug” forms can also be utilized which, when delivered in such form, convert to the active moiety in vivo. See, e.g., B. Testa and J. Caldwell, “Prodrugs Revisited: The “Ad Hoc” Approach as a Complement to Ligand Design”, Medicinal Research Reviews, 16(3):233-241, ed., John Wiley & Sons (1996).

As described herein, the compounds of formula I and/or salts, prodrugs or tautomers thereof, are delivered in regimens for contraception, therapeutic or prophylactic purposes, as described herein.

The compounds discussed herein also encompass “metabolites” which are unique products formed by processing the compounds useful in the invention by the cell or patient. Desirably, metabolites are formed in vivo.

The compounds useful in this invention are readily prepared by one of skill in the art according to the following schemes from commercially available starting materials or starting materials which can be prepared using literature procedures. These schemes show the preparation of representative compounds of this invention. Variations on these methods, or other methods known in the art can be readily utilized by one of skill in the art given the information provided herein.

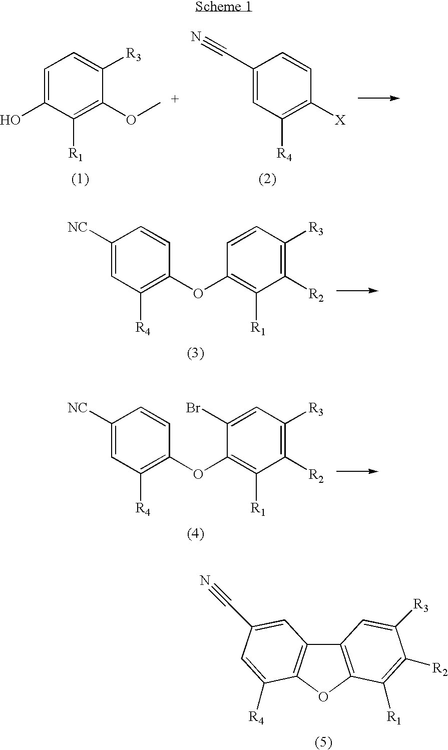

According to Scheme 1, an appropriately substituted phenol (1) is treated with a suitable base and an appropriately substituted aryl halide (2; X=halogen) to afford diphenyl ethers (3). Generally the compounds of the invention are prepared using KF-alumina or potassium carbonate as the base, in suitable solvents such as acetonitrile or dimethyl sulfoxide, or another polar solvent. In the case of KF-alumina, the base may also be used in conjunction with an additive such as a crown ether. The aryl halide (2) is typically an aryl fluoride (X═F), an aryl chloride (X═Cl) or an aryl bromide (X═Br).

Diphenylethers (3) are then brominated to give compound (4). The bromination is generally accomplished with N-bromosuccinimide in a solvent such as acetonitrile.

Compound (4) is then converted into compound (5) under the action of a palladium catalyst in a suitable solvent. The source of palladium is normally palladium acetate or dichlorobis(triphenylphosphine)palladium. In one embodiment, the solvent is selected from dimethylformamide or dimethylacetamide. However, other suitable solvents may readily be selected by one of skill in the art. Where the R₂ group of compound (5) is desired to be hydroxy, the corresponding compound (5) containing a methoxy group is treated with a demethylating agent such as pyridine hydrochloride or boron tribromide.

Pharmaceutical compositions comprising one or more compounds and a pharmaceutically acceptable carrier or excipient may be used in the methods and kits of the invention. The invention also includes methods of treatment which comprise administering to a mammal a pharmaceutically effective amount of one or more compounds as described above as modulators of the progesterone receptor.

The compounds useful in this invention can be utilized in methods of contraception, hormone replacement therapy, cycle-related symptoms and the treatment and/or prevention of benign and malignant neoplastic disease. Specific uses of the compounds and pharmaceutical compositions of invention include the treatment and/or prevention of uterine myometrial fibroids, endometriosis, benign prostatic hypertrophy; carcinomas and adenocarcinomas of the endometrium, ovary, breast, colon, prostate, pituitary, meningioma and other hormone-dependent tumors; dysmenorrhea; dysfunctional uterine bleeding; and symptoms of premenstrual syndrome and premenstrual dysphoric disorder; and for inducing amenorrhea. Additional uses of the present progesterone receptor modulators include the synchronization of the estrus in livestock.

The term “cycle-related symptoms” refers to psychological symptoms (for example, mood change, irritability, anxiety, lack of concentration, or decrease in sexual desire) and physical symptoms (for example, dysmenorrhea, breast tenderness, bloating, fatigue, or food cravings) associated with a woman's menstrual cycle. Cycle-related symptoms occur after ovulation but before menses and usually terminate at the start of the menstrual period or shortly thereafter. Cycle-related symptoms include, but are not limited to, dysmenorrhea and moderate to severe cycle-related symptoms.

Suitably, the PR modulators used in the invention are formulated for delivery by any suitable route including, e.g., transdermal, mucosal (intranasal, buccal, vaginal), oral, parenteral, etc., by any suitable delivery device including, e.g., transdermal patches, topical creams or gels, a vaginal ring, among others.

When the compounds are employed for the above utilities, they may be combined with one or more pharmaceutically acceptable carriers or excipients, for example, solvents, diluents and the like, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solutions or suspensions containing from about 0.05 to 5% suspending agent in an isotonic medium. Such pharmaceutical preparations may contain, for example, from about 25 to about 90% of the active ingredient in combination with the carrier, more usually between about 5% and 60% by weight.

The effective dosage of active ingredient employed may vary depending on the particular compound employed, the mode of administration and the severity of the condition being treated. However, in general, satisfactory results are obtained when the compounds of the invention are administered at a daily dosage of from about 0.5 to about 500 mg/kg of animal body weight, desirably given in divided doses one to four times a day, or in a sustained release form. For most large mammals, the total daily dosage is from about 1 to 100 mg, desirably from about 2 to 80 mg. Dosage forms suitable for internal use comprise from about 0.5 to 500 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

These active compounds may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

The pharmaceutical compositions from the standpoint of ease of preparation and administration are solid compositions, particularly tablets and hard-filled or liquid-filled capsules. Oral administration of the compounds is desirable.

These active compounds may also be administered parenterally or intraperitoneally. Solutions or suspensions of these active compounds as a free base or pharmacologically acceptable salt can be prepared in water suitably mixed with a surfactant such as hydroxypropylcellulose. Dispersions can also be prepared in glycerol, liquid, polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exits. It must be stable under conditions of manufacture and storage and must be preserved against the contaminating action of microorganisms such as bacteria and fungi. The carrier can be a solvent or dispersion medium containing, for example, water, ethanol (e.g., glycerol, propylene glycol and liquid polyethylene glycol), suitable mixtures thereof, and vegetable oil.

In one embodiment, the present invention provides cyclic regimens involving administration of a PR modulator of the invention alone. In another embodiment, the cyclic regimen involves administration of a PR modulator of the invention in combination with an estrogen or progestin, or both. Particularly desirable progestins can be selected from among those described in U.S. Pat. Nos. 6,355,648; 6,521,657; 6,436,929; 6,540,710; and 6,562,857 and U.S. Patent Application Publication No.2004-0006060-A1. Still other progestins are known in the art and can be readily selected. In one embodiment, the present invention provides combination regimens with the PR agonist (i.e., progestin) 5-(4,4-dimethyl-2-thioxo-1,4-dihydro-2H-3,1-benzoxazin-6-yl)-1-methyl-1H-pyrrole-2-carbonitrile, also known as tanaproget.

This invention further includes administration regimens carried out over 28 consecutive days. These regimens may be continuous, or may involve a terminal portion of the cycle, e.g., 0 to 7 days, containing administration of no progestins, estrogens or anti-progestins. See, e.g., the regimens described in U.S. patent application No. 11,175,549, and its international counterpart, PCT/US04/23798, which are hereby incorporated by reference.

The regimens described herein may be utilized for contraception, or for any of the other indications described herein. Where administration is for contraception, the compositions may be formulated in oral dosage units.

When utilized for contraception, the PR modulators used in the invention may be administered to a female of child bearing age, alone or in combination with an estrogen. For the first 14 to 24 days of the cycle, a progestational agent is administered, desirably at a dosage range equal in progestational activity to about 35 μg to about 150 μg levonorgestrel per day, and more desirably equal in activity to about 35 μg to about 100 μg levonorgestrel per day. A PR modulator as described herein may then be administered alone or in combination with an estrogen for a period of 1 to 11 days to begin on any cycle day between day 14 and 24. The PR modulator in these combinations may be administered at a dose of from about 2 μg to about 50 μg per day and the estrogen may be administered at a dose of from about 10 μg to about 35 μg per day. In an oral administration, a package or kit containing 28 tablets will include a placebo tablet on those days when the PR modulator of formula I or progestin or estrogen is not administered.

Progestational agents useful with this invention include, but are not limited to, tanaproget, levonorgestrel, norgestrel, desogestrel, 3-ketodesogestrel, norethindrone, gestodene, norethindrone acetate, norgestimate, osaterone, cyproterone acetate, trimegestone, dienogest, drospirenone, nomegestrol, or (17-deacetyl)norgestimate. Among the desirable progestins for use in the combinations of this invention are levonorgestrel, gestodene and trimegestone.

Examples of orally administered regimens of this invention over a 28 day cycle include administration of a progestational agent solely for the first 21 days at a daily dose equal in progestational activity to from about 35 to about 100 μg of levonorgestrel. A PR modulator compound of formula I can then be administered at a daily dose of from about 1 to 200 mg from day 22 to day 24, followed by no administration or administration of a placebo for days 25 to 28. It is most desirable that the daily dosages of each relevant active ingredient be incorporated into a combined, single daily dosage unit, totaling 28 daily units per 28-day cycle.

In another regimen, a progestational agent may be coadministered for the first 21 days at a daily dose equal in progestational activity to from about 35 to about 150 μg levonorgestrel, desirably equal in activity to from about 35 to about 100 μg levonorgestrel, with an estrogen, such as ethinyl estradiol, at a daily dose range of from about 10 to about 35 μg. This may be followed as described above with a PR modulator of the invention administered at a daily dose of from about 1 to 250 mg from day 22 to day 24, followed by no administration or administration of a placebo for days 25 to 28.

Still another regimen within the scope of this invention will include coadministration from days 1 to 21 of a progestational agent, e.g., levonorgestrel, being administered at a daily dose equal in progestational activity to from about 35 to about 100 μg levonorgestrel, and an estrogen, such as ethinyl estradiol, at a daily dose range of from about 10 to about 35 μg. This will be followed on days 22 to 24 by coadministration of a PR modulator of the invention (1 to 250 mg/day) and an estrogen, such as ethinyl estradiol, at a daily dose of from about 10 to about 35 μg. From day 25 to day 28, this regimen may be followed by no administration or administration of a placebo.

This invention also includes kits or packages of pharmaceutical formulations designed for use in the regimens described herein. These kits are desirably designed for daily oral administration over a 28-day cycle, desirably for one oral administration per day, and organized so as to indicate a single oral formulation or combination of oral formulations to be taken on each day of the 28-day cycle. Desirably, each kit will include oral tablets to be taken on each of the days specified, desirably one oral tablet will contain each of the combined daily dosages indicated.

In each of the regimens, kits, and packages just described, it is preferred that the daily dosage of each pharmaceutically active component of the regimen remain fixed in each particular phase in which it is administered. It is also understood that the daily dose units described are to be administered in the order described, with the first phase followed in order by the second and third phases. To help facilitate compliance with each regimen, it is also preferred that the kits contain the placebo described for the final days of the cycle. It is further preferred that each package or kit comprise a pharmaceutically acceptable package having indicators for each day of the 28-day cycle, such as a labeled blister package or dial dispenser package known in the art.

These dosage regimens may be adjusted to provide the optimal therapeutic response. For example, several divided doses of each component may be administered daily or the dose may be proportionally increased or reduced as indicated by the exigencies of the therapeutic situation. In the descriptions herein, reference to a daily dosage unit may also include divided units which are administered over the course of each day of the cycle contemplated.

According to the regimens described above, one 28-day kit may comprise (a) an initial phase of from 14 to 21 daily dosage units of a progestational agent equal in progestational activity to about 35 to about 150 μg levonorgestrel, desirably equal in progestational activity to about 35 to about 100 μg levonorgestrel; (b) a second phase of from 1 to 11 daily dosage units of a PR modulator compound of formula I, each daily dosage unit containing the PR modulator compound at a daily dosage of from about 1 to 250 mg; and (c) optionally, a third phase of an orally and pharmaceutically acceptable placebo for the remaining days of the cycle in which no PR modulator (i.e., antiprogestin or progestin) or estrogen is administered.

In one embodiment of this kit, the initial phase involves 21 daily dosage units as described in the preceding passage, a second phase of 3 daily dosage units for days 22 to 24 of a PR modulator compound of formula I and an optional third phase of 4 daily units of an orally and pharmaceutically acceptable placebo for each of days 25 to 28.

In another embodiment, a 28-day cycle packaging regimen or kit of this invention contains, a first phase of from 18 to 21 daily dosage units, and more desirably, 21 days, as described in the preceding passages, and, further including, as an estrogen, ethinyl estradiol at a daily dose range of from about 10 to about 35 μg; a second phase of from 1 to 7 daily dosage units, and desirably, 4 daily dosage units, as described above, and an optional placebo for each of the remaining 0-9 days, or about 4 days, in the 28-day cycle in which no progestational agent, estrogen or antiprogestin is administered.

A further 28-day packaged regimen or kit of this invention comprises (a) a first phase of from 18 to 21 daily dosage units, each containing a progestational agent at a daily dose equal in progestational activity to about 35 to about 150 μg levonorgestrel, desirably equal in activity to from about 35 to about 100 μg levonorgestrel, and ethinyl estradiol at a daily dose range of from about 10 to about 35 μg; (b) a second phase of from 1 to 7 daily dose units, each daily dose unit containing a PR modulator of this invention at a concentration of from 1 to 250 mg and ethinyl estradiol at a concentration of from about 10 to about 35 μg; and (c) optionally, an orally and pharmaceutically acceptable placebo for each of the remaining 0-9 days in the 28-day cycle in which no progesiational agent, estrogen or antiprogestin is administered.

In one embodiment, the package or kit just described comprises a first phase of 21 daily dosage units; a second phase of 3 daily dosage units for days 22 to 24, each daily dose unit containing an PR modulator of formula I at a concentration of from 2 to 200 mg and ethinyl estradiol at a concentration of from about 10 to about 35 μg; and optionally, a third phase of 4 daily units of an orally and pharmaceutically acceptable placebo for each of days 25 to 28.

In each of the regimens and kits just described, it is desirable that the daily dosage of each pharmaceutically active component of the regimen remain fixed in each particular phase in which it is administered. It is also understood that the daily dose units described are to be administered in the order described, with the first phase followed in order by the second and third phases. To help facilitate compliance with each regimen, it is also desirable that the kits contain the placebo described for the final days of the cycle. It is further desirable that each package or kit comprise a pharmaceutically acceptable package having indicators for each day of the 28-day cycle, such as a labeled blister package or dial dispenser packages known in the art.

As used herein, the terms anti-progestational agents, anti-progestins and progesterone receptor antagonists are understood to be synonymous. Similarly, progestins, progestational agents and progesterone receptor agonists are understood to refer to compounds of the same activity.

These dosage regimens may be adjusted to provide the optimal therapeutic response. For example, several divided doses of each component may be administered daily or the dose may be proportionally increased or reduced as indicated by the exigencies of the therapeutic situation. In the descriptions herein, reference to a daily dosage unit may also include divided units which are administered over the course of each day of the cycle contemplated.

The desirable pharmaceutical compositions from the standpoint of ease of preparation and administration are solid compositions, particularly tablets and hard-filled or liquid-filled capsules. Oral administration of the compounds is desirable.

These active compounds may also be administered via a vaginal ring. Suitably, use of the vaginal ring is timed to the 28 day cycle. In one embodiment, the ring is inserted into the vagina, and it remains in place for 3 weeks. During the fourth week, the vaginal ring is removed and menses occurs. The following week a new ring is inserted to be worn another 3 weeks until it is time for the next period. In another embodiment, the vaginal ring is inserted weekly, and is replaced for three consecutive weeks. Then, following one week without the ring, a new ring is inserted to begin a new regimen. In yet another embodiment, the vaginal ring is inserted for longer or shorter periods of time.

For use in the vaginal ring, a PR modulator compound is formulated in a manner similar to that described for contraceptive compounds previously described for delivery via a vaginal ring. See, e.g., U.S. Pat. Nos. 5,972,372; 6,126,958 and 6,125,850.

In still another aspect of the invention, the PR modulator compound(s) are delivered via a transdermal patch. Suitably, use of the patch is timed to the 28 day cycle. In one embodiment, the patch is applied via a suitable adhesive on the skin, where it remains in place for 1 week and is replaced weekly for a total period of three weeks. During the fourth week, no patch is applied and menses occurs. The following week a new patch is applied to be worn to begin a new regimen. In yet another embodiment, the patch remains in place for longer, or shorter periods of time.

The invention further provides for kits and delivery devices containing the compounds of the invention for a variety of other therapeutic uses as described herein including, e.g., hormone replacement therapy, the treatment and/or prevention of benign and malignant neoplastic disease. Such kits contain components in addition to the compounds of the invention, including, e.g., instructions for delivery of the compounds of the invention, diluents, vials, syringes, packaging, among other items.

Such kits may optionally be adapted for the selected application, e.g., hormone replacement therapy, cycle-related symptoms, treatment and/or prevention of uterine myometrial fibroids, endometriosis, benign prostatic hypertrophy; carcinomas and adenocarcinomas of the endometrium, ovary, breast, colon, prostate, pituitary, meningioma and other hormone-dependent tumors, or the synchronization of the estrus in livestock.

The following examples are provided to illustrate the invention and do not limit the scope thereof. One skilled in the art will appreciate that although specific reagents and conditions are outlined in the following examples, modifications can be made which are meant to be encompassed by the spirit and scope of the invention.

### EXAMPLE 1

### 7-methoxydibenzo[b,d]furan-2-carbonitrile

### A. 4-(2-bromo-5-methoxyphenoxy)benzonitrile

A mixture of 4-(3-methoxyphenoxy)benzonitrile (Sawyer, J. Scott; Schmittling, Elisabeth A.; Palkowitz, Jayne A.; Smith, William J., III. Journal of Organic Chemistry (1998), 63(18), 6338-6343) (1.39 g, 6.17 mmol) and N-bromosuccinimide (1.21 g, 6.79 mmol) in acetonitrile (30 mL) was stirred at room temperature for 24 h. The solution was poured into a mixture of 50 mL diethyl ether and 50 mL water, the organic layer was washed with brine, dried over magnesium sulfate, and concentrated. The residue was purified by silica gel column chromatography (hexane/ethyl acetate, 95/5 to 90/10) to afford a mixture of 4-(2-bromo-5-methoxyphenoxy)benzonitrile and 4-(4-bromo-3-methoxyphenoxy)benzonitrile which was further purified by reversed-phase preparative liquid chromatography (water/acetonitrile) to afford 4-(2-bromo-5-methoxyphenoxy)benzonitrile (0.99 g, 53%) as an orange oil.

### B. 7-methoxydibenzo[b,d]furan-2-carbonitrile

A mixture of 4-(2-bromo-5-methoxyphenoxy)benzonitrile (0.99 g, 3.3 mmol), palladium acetate (0.365 g, 1.63 mmol), and sodium carbonate (0.5 g) in dimethylacetamide (10 mL) was heated at reflux under a nitrogen atmosphere for 18 h. The mixture was then cooled and 150 mL of water was added. The mixture was filtered through a plug of CELITE™ gel, and the CELITE™ gel was rinsed with 100 mL of water followed by 300 mL of ethyl ether, and the filtrates combined. The aqueous layer was extracted twice with ethyl ether, and the combined organic layers were washed with brine, dried over magnesium sulfate, and concentrated. The residue was purified by reversed-phase preparative liquid chromatography (water/acetonitrile) to afford 7-methoxydibenzo[b,d]furan-2-carbonitrile (220 mg, 30%) as a white solid.

MS (EI) m/z 223.1; HPLC purity 98.4% (210-370 nm, no impurities detected@306 nm; RT=10.0 min; Xterra RP18, 3.5u, 4.6×150 mm column, 1.2 mL/min, 85/15-5/95 (Ammon. Form. Buff. Ph=3.5/ACN+MeOH) for 10 min, hold 4 min.

### EXAMPLE 2

### 7-hydroxydibenzo[b,d]furan-2-carbonitrile

A mixture of 7-methoxydibenzo[b,d]furan-2-carbonitrile (50 mg, 0.22 mmol) [Example 1] and pyridine hydrochloride (388 mg, 3.4 mmol) was heated in a sealed tube at 185° C. for 3 h. The mixture was cooled, diluted with water, neutralized with aqueous sodium hydroxide, and extracted twice with ethyl acetate. The combined organic layers were washed with brine, dried over magnesium sulfate, and concentrated. The residue was purified by silica gel column chromatography (hexane/ethyl acetate, 90/10 to 80/20) to afford 7-hydroxydibenzo[b,d]furan-2-carbonitrile (32 mg, 68%) as a white solid.

mp 270-271° C.; MS (ESI) m/z 208; HPLC purity: The major component is 98.4% at 210-370 nm window; and 96.8% at 240 nm@max. abs. RT=9.0 min; Xterra RPI 8, 3.5u, 150×4.6 mm column, 1.2 mL/min, 85/15-5/95 (Ammon. Form. Buff. pH=3.5/ACN+MeOH) for 10 min, hold 4 min.

### EXAMPLE 3

### 6-bromo-8-fluoro-7-hydroxydibenzo[b,d]furan-2-carbonitrile

A solution of 8-fluoro-7-hydroxydibenzo[b,d]furan-2-carbonitrile (0.790 g, 3.48 mmol) and N-bromosuccinimide (0.492 g, 4.17 mmol) in anhydrous tetrahydrofuran was stirred under a nitrogen atmosphere at room temperature for 10 min. The solvent was evaporated and the residue purified by silica gel column chromatography (hexane/ethyl acetate, 9/1 to 1/1) to afford 6-bromo-8-fluoro-7-hydroxydibenzo[b,d]furan-2-carbonitrile (0.600 g, 56%) as a white solid.

mp 270° C. (dec); MS (ES) m/z 303.9; HPLC purity: no impurities detected at 210-370 nm window; and no impurities detected at 300 nm@max. abs.; Xterra RPI 8, 3.5u, 150×4.6 mm column, 1.2 mL/min, 85/15-5/95 (Ammon. Form. Buff. Ph=3.5/ACN+MeOH) for 10 min, hold 4 min. HRMS: calcd for C₁₃H₅BrFNO₂—H+, 303.94149; found (ESI, [M-H]⁻), 303.94.

### EXAMPLE 4

### 8-fluoro-7-hydroxy-6-(2-methylphenyl)dibenzo[b,d]furan-2-carbonitrile

A mixture of 6-bromo-8-fluoro-7-hydroxydibenzo[b,d]furan-2-carbonitrile (48 mg, 0.16 mmol), o-tolylboronic acid (21 mg, 0.16 mmol), tetrakis(triphenylphosphine)palladium(0) (18 mg, 0.016 mmol), and aqueous sodium carbonate (0.16 mL of a 2M solution, 0.32 mmol) in dimethoxyethane (2.5 mL) was heated at reflux for 16 h. The mixture was cooled and partitioned between water and ethyl acetate, and the aqueous layer was extracted twice with ethyl acetate. The combined organic layers were washed with brine, dried over magnesium sulfate, and concentrated. The residue was purified by reversed-phase preparative liquid chromatography (water/acetonitrile) to afford 8-fluoro-7-hydroxy-6-(2-methylphenyl)dibenzo[b,d]furan-2-carbonitrile (7 mg, 13%) as a yellow oil.

MS (ES) m/z 316.1; HPLC purity Major Component=96.6% at 210-370 nm window; and =98.1% at 310 nm (max. abs.; Xterra RP18, 3.5u, 150×4.6 mm column, 1.2 mL/min, 85/15-5/95 (Ammon. Form. Buff. Ph=3.5/ACN+MeOH) for 10 min, hold 4 min. HRMS: calcd for C₂₀H₁₂FNO₂—H+, 316.07793; found (ESI, [M-H]⁻), 316.0786.

### EXAMPLE 5

### 6-(2-chlorophenyl)-8-fluoro-7-hydroxydibenzo[b,d]furan-2-carbonitrile

A mixture of 6-bromo-8-fluoro-7-hydroxydibenzo[b,d]furan-2-carbonitrile (48 mg, 0.16 mmol), 2-chlorophenylboronic acid (25 mg, 0.16 mmol), tetrakis(triphenylphosphine)palladium(0) (18 mg, 0.016 mmol), and aqueous sodium carbonate (0.16 mL of a 2M solution, 0.32 mmol) in dimethoxyethane (2.5 mL) was heated at reflux for 16 h. The mixture was cooled and partitioned between water and ethyl acetate, and the aqueous layer was extracted twice with ethyl acetate. The combined organic layers were washed with brine, dried over magnesium sulfate, and concentrated. The residue was purified by reversed-phase preparative liquid chromatography (water/acetonitrile) to afford 8-fluoro-7-hydroxy-6-(2-chlorophenyl)dibenzo[b,d]furan-2-carbonitrile (10 mg, 20%) as a yellow oil.

MS (ES) m/z 336.1; HPLC purity Major Component=90.5% at 210-370 nm window; and =92.9% at 310 nm (max. abs.; Xterra RP18, 3.5u, 150×4.6 mm column, 1.2 mL/min, 85/15-5/95 (Ammon. Form. Buff. pH=3.5/ACN+MeOH) for 10 min, hold 4 min. HRMS: calcd for C₁₉H₉ClFNO₂—H+, 336.02331; found (ESI, [M-H]⁻), 336.0215.

### EXAMPLE 6

### 8-fluoro-7-hydroxydibenzo[b,d]furan-2-carbonitrile

A mixture of 8-fluoro-7-methoxydibenzo[b,d]furan-2-carbonitrile (4.40 g, 18.2 mmol) and pyridine hydrochloride (31.6 g, 274 mmol) was heated in a sealed tube at 195° C. for 3 h. The mixture was cooled, diluted with water, neutralized with aqueous sodium hydroxide, and extracted twice with ethyl acetate. The combined organic layers were washed with brine, dried over magnesium sulfate, and concentrated. The residue was purified by reversed-phase preparative liquid chromatography (water/acetonitrile) to afford 8-fluoro-7-hydroxydibenzo[b,d]furan-2-carbonitrile (2.1 g, 51%) as a white solid.

mp 270° C. (dec); MS (ES) m/z 226.0; HPLC purity Major Component=99.6% at 210-370 nm window; and no impurities detected at 310 nm@max. abs.; Xterra RP18, 3.5u, 150×4.6 mm column, 1.2 mL/m in, 85/15-5/95 (Ammon. Form. Buff. Ph=3.5/ACN+MeOH) for 10 min, hold 4 min. HRMS: calcd for C₁₃H₆FNO₂—H+, 226.03098; found (ESI, [M-H]⁻), 226.0315.

### EXAMPLE 7

### 8-fluoro-7-methoxydibenzo[b,d]furan-2-carbonitrile

### A. 4-(4-fluoro-3-methoxyphenoxy)benzonitrile

This compound was prepared according to the method of Sawyer, et al. (Sawyer, J. Scott; Schmittling, Elisabeth A.; Palkowitz, Jayne A.; Smith, William J., III. Journal of Organic Chemistry (1998), 63(18), 6338-6343) from 4-fluoro-3-methoxyphenol (Belanger, Patrice C.; Lau, C. K.; Williams, Haydn W. R.; Dufresne, C.; Scheigetz, John. Canadian Journal of Chemistry (1988), 66(6), 1479-82) (9.71 g, 68.3 mmol) and 4-fluorobenzonitrile (8.27 g, 68.3 mmol), which provided 4-(4-fluoro-3-methoxyphenoxy)benzonitrile (11.69 g, 70%) as an orange oil.

### B. 4-(2-bromo-4-fluoro-5-methoxyphenoxy)benzonitrile

A mixture of 4-(4-fluoro-3-methoxyphenoxy)benzonitrile (11.69 g, 48.1 mmol) and N-bromosuccinimide (17.01 g, 144.2 mmol) in acetonitrile (100 mL) was stirred at room temperature for 16 h. The solution was poured into a mixture of 150 mL diethyl ether and 150 mL water, the organic layer was washed with brine, dried over magnesium sulfate, and concentrated. The residue was purified by silica gel column chromatography (hexane/ethyl acetate, 95/5 to 50/50) to afford a 4-(2-bromo-4-fluoro-5-methoxyphenoxy)benzonitrile (14.04 g, 91%) as a yellow oil.

### C. 8-fluoro-7-methoxydibenzo[b,d]furan-2-carbonitrile

A mixture of 4-(2-bromo-4-fluoro-5-methoxyphenoxy)benzonitrile (14.04 g, 43.6 mmol), palladium acetate (0.979 g, 4.36 mmol), and sodium carbonate (7 g) in dimethylacetamide (150 mL) was heated at reflux under a nitrogen atmosphere for 16 h. The mixture was then cooled and 300 mL of water and 300 mL ethyl ether were added. The aqueous layer was extracted seven times with ethyl acetate, and the combined organic layers were washed with brine, dried over magnesium sulfate, and concentrated. The residue was purified by silica gel column chromatography (hexane/ethyl acetate, 90/10) to afford 8-fluoro-7-methoxydibenzo[b,d]furan-2-carbonitrile (5.1 g, 48%) as a white solid.

mp 206-208° C.; Anal. Calcd for C14H8FNO2: C, 69.71; H, 3.34; N, 5.81. Found: C, 69.8; H, 3.52; N, 5.59; HPLC purity Major Component=99.1% at 210-370 nm window; and no impurities detected at 304 nm@max. abs.; Xterra RP18, 3.5u, 150×4.6 mm column, 1.2 mL/min, 85/15-5/95 (Ammon. Form. Buff. Ph=3.5/ACN+MeOH) for 10 min, hold 4 min.

### EXAMPLE 8

### 8-fluoro-6-(2-fluorophenyl)-7-hydroxydibenzo[b,d]furan-2-carbonitrile

A mixture of 6-bromo-8-fluoro-7-hydroxydibenzo[b,d]furan-2-carbonitrile (48 mg, 0.16 mmol), 2-fluorophenylboronic acid (22 mg, 0.16 mmol), tetrakis(triphenylphosphine)palladium(0) (18 mg, 0.016 mmol), and aqueous sodium carbonate (0.16 mL of a 2M solution, 0.32 mmol) in dimethoxyethane (2.5 mL) was heated at reflux for 16 h. The mixture was cooled and partitioned between water and ethyl acetate, and the aqueous layer was extracted twice with ethyl acetate. The combined organic layers were washed with brine, dried over magnesium sulfate, and concentrated. The residue was purified by reversed-phase preparative liquid chromatography (water/acetonitrile) to afford 8-fluoro-7-hydroxy-6-(2-fluorophenyl)dibenzo[b,d]furan-2-carbonitrile (11 mg, 22%) as a yellow oil.

MS (ESI) m/z 320; HPLC purity Major=92.7% at 210-370 nm window; and =93.3% at 310 nm@max. abs. RT=9.9 min; Xterra RP18, 3.5u, 150×4.6 mm column, 1.2 mL/min, 85/15-5/95 (Ammon. Form. Buff. Ph=3.5/ACN+MeOH) for 10 min, hold 4 min.

### EXAMPLE 9

### 6-isopropyl-7-methoxydibenzo[b,d]furan-2-carbonitrile

### A. 4-(2-isopropyl-3-methoxyphenoxy)benzonitrile

This compound was prepared according to the method of Li, et al. (Li, F.; Wang, Q.; Ding, Z.; Tao, F. Org. Lett. (2003), 5(12), 2169-2171) from 2-isopropyl-3-methoxyphenol (Engler, Thomas A.; Sampath, Umashanker; Naganathan, Sriram; Vander Velde, David; Takusagawa, Fusao; Yohannes, Daniel. Journal of Organic Chemistry (1989), 54(24), 5712-27) (0.57 g, 3.4 mmol) and 4-fluorobenzonitrile (0.42 g, 3.4 mmol), which provided 4-(2-isopropyl-3-methoxyphenoxy)benzonitrile (0.70 g, 76%) as a yellow oil.

### B. 4-(6-bromo-2-isopropyl-3-methoxyphenoxy)benzonitrile

A mixture of 4-(2-isopropyl-3-methoxyphenoxy)benzonitrile (0.70 g, 2.6 mmol) and N-bromosuccinimide (0.34 g, 2.9 mmol) in acetonitrile (20 mL) was stirred at room temperature for 2 h. The solution was poured into a mixture of 25 mL diethyl ether and 25 mL water, the organic layer was washed with brine, dried over magnesium sulfate, and concentrated. The residue was purified by silica gel column chromatography (hexane/ethyl acetate, 100/0 to 80/20) to afford a 4-(6-bromo-2-isopropyl-3-methoxyphenoxy)benzonitrile (0.70 g, 77%) as a yellow oil.

### C. 6-isopropyl-7-methoxydibenzo[b,d]furan-2-carbonitrile

A mixture of 4-(6-bromo-2-isopropyl-3-methoxyphenoxy)benzonitrile (0.70 g, 2.0 mmol), palladium acetate (0.227 g, 1.0 mmol), and sodium carbonate (0.35 g) in dimethylacetamide (20 mL) was heated at reflux under a nitrogen atmosphere for 16 h. The mixture was then cooled and 200 mL of water and 200 mL ethyl ether were added. The aqueous layer was extracted twice with ethyl ether, and the combined organic layers were washed with brine, dried over magnesium sulfate, and concentrated. The residue was purified by reversed-phase preparative liquid chromatography (water/acetonitrile) to afford 6-isopropyl-7-methoxydibenzo[b,d]furan-2-carbonitrile (0.160 g, 30%) as a yellow oil.

MS m/z 266; HPLC purity no imp. detect. at 210-370 nm window; and no imp. detect. at 304 nm (max. abs) RT=11.4; Xterra RPI 8, 3.5u, 150×4.6 mm column, 1.2 mL/min, 85/15-5/95 (Ammon. Form. Buff. Ph=3.5/ACN+MeOH) for 10 min, hold 4 min. HRMS: calcd for C₁₇H₁₅NO₂, 265.11028; found (El, M+.), 265.1104.

### EXAMPLE 10

### 7-hydroxy-6-isopropyidibenzo[b,d]furan-2-carbonitrile

A solution of 6-isopropyl-7-methoxydibenzo[b,d]furan-2-carbonitrile (0.160 g, 0.60 mmol) and boron tribromide (1.92 mL of a IM solution in dichloromethane, 1.92 mmol) in dichloromethane (50 mL) was stirred under a nitrogen atmosphere at room temperature for 16 h. Water (50 mL) was added, and the aqueous layer was extracted twice with dichloromethane. The combined organic layers were washed with brine, dried over magnesium sulfate, and concentrated. The residue was purified by silica gel column chromatography (hexane/ethyl acetate, 90/10 to 70/30) to afford 7-hydroxy-6-isopropyldibenzo[b,d]furan-2-carbonitrile (0.140 g, 93%) as a white solid.

mp 180-182° C.; MS (ESI) m/z 250; HPLC purity no imp. detect. at 210-370 nm window; and no imp. detect. at 304 nm (max. abs) RT=10.7; Xterra RP18, 3.5u, 150×4.6 mm column, 1.2 mL/min, 85/15-5/95 (Ammon. Form. Buff. Ph=3.5/ACN+MeOH) for 10 min, hold 4 min. HRMS: calcd for C₁₆H₁₃NO₂—H+, 250.08735; found (ESI, [M-H]⁻), 250.0867.

### EXAMPLE 11

### 6-cyclopentyl-7-hydroxydibenzo[b,d]furan-2-carbonitrile

### A. 4-(2-cyclopentyl-3-methoxyphenoxy)benzonitrile

This compound was prepared according to the method of Li, et al. (Li, F.; Wang, Q.; Ding, Z.; Tao, F. Org. Lett. (2003), 5(12), 2169-2171) from 2-cyclopentyl-3-methoxyphenol (Yusupov, A.; Abdurasuleva, A. R. Doklady Akademii Nauk USSR (1970), 27(6), 38-9) (1.36 g, 7.07 mmol) and 4-fluorobenzonitrile (0.856 g, 7.07 mmol), which provided 4-(2-cyclopentyl-3-methoxyphenoxy)benzonitrile (1.9 g, 92%) as a yellow oil.

### B. 4-(6-bromo-2-cyclopentyl-3-methoxyphenoxy)benzonitrile

A mixture 4-(2-cyclopentyl-3-methoxyphenoxy)benzonitrile (1.71 g, 5.83 mmol) and N-bromosuccinimide (0.894 g, 7.58 mmol) in acetonitrile (40 mL) was stirred at room temperature for 2 h. The solution was poured into a mixture of 50 mL diethyl ether and 50 mL water, the organic layer was washed with brine, dried over magnesium sulfate, and concentrated. The residue was purified by silica gel column chromatography (hexane/ethyl acetate, 95/5 to 90/10) to afford a 4-(6-bromo-2-cyclopentyl-3-methoxyphenoxy)benzonitrile (0.62 g, 29%) as a yellow oil.

### C. 6-cyclopentyl-7-methoxydibenzo[b,d]furan-2-carbonitrile

A mixture of 4-(6-bromo-2-cyclopentyl-3-methoxyphenoxy)benzonitrile (0.620 g, 1.67 mmol), palladium acetate (0.187 g, 0.83 mmol), and sodium carbonate (0.35 g) in dimethylacetamide (20 mL) was heated at reflux under a nitrogen atmosphere for 16 h. The mixture was then cooled and 200 mL of water and 200 mL ethyl ether were added. The aqueous layer was extracted twice with ethyl ether, and the combined organic layers were washed with brine, dried over magnesium sulfate, and concentrated. The residue was purified by reversed-phase preparative liquid chromatography (water/acetonitrile) to 6-cyclopentyl-7-methoxydibenzo[b,d]furan-2-carbonitrile (30 mg, 6%) as a yellow oil.

### D. 6-cyclopentyl-7-hydroxydibenzo[b,d]furan-2-carbonitrile

A solution of 6-cyclopentyl-7-methoxydibenzo[b,d]furan-2-carbonitrile (60 mg, 0.21 mmol) and boron tribromide (1.6 mL of a IM solution in dichloromethane, 1.6 mmol) in dichloromethane (10 mL) was stirred under a nitrogen atmosphere at room temperature for 16 h. Water (10 mL) was added, and the aqueous layer was extracted twice with dichloromethane. The combined organic layers were washed with brine, dried over magnesium sulfate, and concentrated. The residue was purified by reversed-phase preparative liquid chromatography (water/acetonitrile) to afford 6-cyclopentyl-7-hydroxydibenzo[b,d]furan-2-carbonitrile (50 mg, 88%) as a white solid.

MS (ESI) m/z 276; HPLC purity 97.0% at 210-370 nm, 11.3 min.; 100% at 306 nm, 11.3 min.; Xterra RPI 8, 3.5u, 150×4.6 mm column, 1.2 mL/min, 85/15-5/95 (Ammon. Form. Buff. Ph=3.5/ACN+MeOH) for 10min, hold 4 min. HRMS: calcd for C₁₈H₁₅NO₂—H+, 276.10300; found (ESI, [M-H]⁻), 276.1033.

### EXAMPLE 12

### 6-sec-butyl-7-methoxydibenzo[b,d]furan-2-carbonitrile

### A. 2-(2, 6-dimethoxyphenyl)-butan-2-ol

To a solution of 1,3-dimethoxybenzene (15.41 g, 112 mmol) in diethyl ether (320 mL) was added n-butyllithium (2.5M in hexane, 49 mL, 123 mmol) and the mixture was heated at reflux for 5 h, then cooled to −78° C. 2-butanone was added, and the mixture was slowly warmed to room temperature and stirred for 16 h. The reaction was quenched by the addition of water and 2N hydrochloric acid, and extracted three times with ethyl acetate. The combined organic layers were washed with brine, dried over sodium sulfate, and concentrated. The residue was purified by silica gel column chromatography (hexane/ethyl acetate, 90/10 to 85/15) to afford 2-(2,6-dimethoxyphenyl)-butan-2-ol (14.75 g, 63%) as a colorless oil.

### B. 2-sec-butyl-1,3-dimethoxybenzene

A mixture of 2-(2,6-dimethoxyphenyl)-butan-2-ol (5.16 g, 24.6 mmol) and p-toluenesulfonic acid monohydrate (0.468 g, 2.46 mmol) in benzene (98 mL) were heated at 60° C. for 5 min. The mixture was cooled and diluted with hexane, and filtered through a short plug of silica gel. The silica gel was washed with 5% ethyl acetate/hexane, and the eluent was concentrated to afford 4.45 g of a colorless oil which was used without further purification. A mixture of this oil (4.23 g) and palladium on carbon (10%, 1.16 g) in ethyl acetate (220 mL) was stirred under an atmosphere of hydrogen for 96 h. The mixture was filtered through CELITE™ gel and the filtrate concentrated to give 2-sec-butyl-1,3-dimethoxybenzene (4.24 g, 94% over two steps) as a colorless oil.

### C. 2-sec-butyl-3-methoxyphenol

To a solution of 2-sec-butyl-1,3-dimethoxybenzene (4.14 g, 21.3 mmol) in dichloromethane (16 mL) at −78° C. was added boron tribromide (IM in dichloromethane, 16 mL, 16 mmol) and the mixture was slowly warmed to room temperature and stirred for 3 h. The reaction was quenched with water and the dichloromethane removed in vacuo. The residue was extracted three times with ethyl acetate, and the combined organic layers were washed with brine, dried over sodium sulfate, and concentrated. The residue was purified by silica gel column chromatography (hexane/ethyl acetate, 99/1 to 91/9) to afford 2-sec-butyl-3-methoxyphenol (2.70 g, 70%) as a colorless oil.

### D. 4-(2-sec-butyl-3-methoxyphenoxy)benzonitrile

This compound was prepared according to the method of Li, et al. (Li, F.; Wang, Q.; Ding, Z.; Tao, F. Org. Lett. (2003), 5(12), 2169-2171) from 2-sec-butyl-3-methoxyphenol (2.32 g, 12.9 mmol) and 4-fluorobenzonitrile (1.56 g, 12.9 mmol), which provided 4-(2-sec-butyl-3-methoxyphenoxy)benzonitrile (3.49 g, 92%) as a colorless oil.

### E. 4-(6-bromo-2-sec-butyl-3-methoxyphenoxy)benzonitrile

A mixture 4-(2-sec-butyl-3-methoxyphenoxy)benzonitrile (2.75 g, 9.78 mmol) and N-bromosuccinimide (1.92 g, 10.8 mmol) in acetonitrile (98 mL) was stirred at room temperature for 45 h. FLORISIL™ gel is added and the mixture is concentrated. The residue was purified by silica gel column chromatography (hexane/ethyl acetate, 99/1 to 98/2) to afford a 4-(6-bromo-2-sec-butyl-3-methoxyphenoxy)benzonitrile (2.84 g, 81%) as a colorless oil.

### F. 6-sec-butyl-7-methoxydibenzo[b,d]furan-2-carbonitrile

A mixture of 4-(6-bromo-2-sec-butyl-3-methoxyphenoxy)benzonitrile (2.59 g, 7.21 mmol), dichlorobis(triphenylphosphine)palladium(II) (3.04 g, 4.33 mmol), and sodium acetate (1.77 g) in dimethylacetamide (145 mL) was heated at reflux under a nitrogen atmosphere for 24 h. The mixture was concentrated, and the residue was purified by silica gel column chromatography (hexane/ethyl acetate, 97/3 to 94/6) to afford 6-sec-butyl-7-methoxydibenzo[b,d]furan-2-carbonitrile (1.58 g, 78%) of a white solid.

mp 80-82° C.; MS (EI) m/z 279; HPLC purity 99.1% at 210-370 nm, 11.6 min.; 100% at 304 nm, 11.6 min.; Xterra RP18, 3.5u, 150×4.6 mm column, 1.2 mL/min, 85/15-5/95 (Ammon. Form. Buff. Ph=3.5/ACN+MeOH) for 10 min, hold 4 min. HRMS: calcd for C₁₈H₁₇NO₂, 279.12593; found (El, M+.), 279.1269.

### EXAMPLE 13

### 6-sec-butyl-7-hydroxydibenzo[b,d]furan-2-carbonitrile

A solution of 6-sec-butyl-7-methoxydibenzo[b,d]furan-2-carbonitrile (0.600 g, 2.15 mmol) in dichloromethane (72 mL) under a nitrogen atmosphere was cooled to 0° C. Boron tribromide (6.45 mL of a 1M solution in dichloromethane, 6.45 mmol) was added, and the reaction was allowed to warm to room temperature and stirred for 22 h. Water (4 mL) was added, and the mixture was concentrated. The residue was partitioned between water and ethyl acetate, and the aqueous layer was extracted twice with ethyl acetate. The combined organic layers were washed with brine, dried over sodium sulfate, and concentrated. The residue was purified by silica gel column chromatography (hexane/ethyl acetate, 91/9 to 88/12) to afford 6-sec-butyl-7-hydroxydibenzo[b,d]furan-2-carbonitrile (0.543 g, 95%) as a white solid.

mp 206-207° C.; MS (ESI) m/z 264; HPLC purity 100% at 244 nm, 10.9 min.; 100% at 210-370 nm, 10.9 min.; Xterra RPI8, 3.5u, 150×4.6 mm column, 1.2 mL/min, 85/15-5/95 (Ammon. Form. Buff. Ph=3.5/ACN+MeOH) for 10 min, hold 4 min. HRMS: calcd for C₁₇H₁₅NO₂, 265.11028; found (El, M+.), 265.1104.

### EXAMPLE 14

### (+)-6-sec-butyl-7-hydroxydibenzo[b,d]furan-2-carbonitrile

This compound was isolated from the chiral separation of racemic 6-sec-butyl-7-hydroxydibenzo[b,d]furan-2-carbonitrile (0.435 g) using a CHIRALPAK™ AD-H 250×20 mm column with SFC-CO₂ with 22% methanol at a rate of 55 mL/min. at 35° C., affording 203 mg (47%) of (+)-6-sec-butyl-7-hydroxydibenzo[b,d]furan-2-carbonitrile.

[α]_{D}²⁵=+12° (c=0.0104 g/mL, MEOH); MS (ESI) m/z 266; MS (ESI) m/z 264; HPLC purity 100% at 210-370 nm, 11.0 min.; 100% at 244 nm, 11.0 min.; Xterra RP18, 3.5u, 150×4.6 mm column, 1.2 mL/min, 85/15-5/95 (Ammon. Form. Buff. Ph=3.5/ACN+MeOH) for 10 min, hold 4 min.

### EXAMPLE 15

### (−)-6-sec-butyl-7-hydroxydibenzo[b,d]furan-2-carbonitrile

This compound was isolated from racemic 6-sec-butyl-7-hydroxydibenzo[b,d]furan-2-carbonitrile (0.435 g) using the same chiral separation method as (+)-6-sec-butyl-7-hydroxydibenzo[b,d]furan-2-carbonitrile, affording 205 mg (47%) of (−)-6-sec-butyl-7-hydroxydibenzo[b,d]furan-2-carbonitrile.

[α]D²⁵=−13° (c=0.0103 g/mL, MeOH); MS (ESI) m/z 266; MS (ESI) m/z 264; HPLC purity 100% at 210-370 nm, 10.1 min.; 100% at 244 nm, 10.1 min.; Xterra RP18, 3.5u, 150×4.6 mm column, 1.2 mL/min, 85/15-5/95 (Ammon. Form. Buff. Ph=3.5/ACN+MeOH) for 10 min, hold 4 min.

### EXAMPLE 16

### (+)-6-sec-butyl-7-methoxydibenzo[b,d]furan-2-carbonitrile

This compound was isolated from the chiral separation of racemic 6-sec-butyl-7-methoxydibenzo[b,d]furan-2-carbonitrile (0.251 g) using a CHIRALPAK AS-H, 20×250 mm column with SFC-CO₂ with 5% methanol at a rate of 50 mL/min. at 35° C., affording 99 mg (39%) of (+)-6-sec-butyl-7-methoxydibenzo[b,d]furan-2-carbonitrile. [α]_{D}²⁵=+17° (c=0.0103 g/mL, MeOH); MS (EI) m/z 279; HPLC purity 99.1% at 210-370 nm, 11.6 min.; 98.6% at 244 nm, 11.6 min.; Xterra RP18, 3.5u, 150×4.6 mm column, 1.2 mL/min, 85/15-5/95 (Ammon. Form. Buff. Ph=3.5/ACN+MeOH) for 10 min, hold 4 min.

### EXAMPLE 17

### (−)-6-sec-butyl-7-methoxydibenzo[b,d]furan-2-carbonitrile

This compound was isolated from racemic 6-sec-butyl-7-methoxydibenzo[b,d]furan-2-carbonitrile (0.251 g) using the same chiral separation method as (+)-6-sec-butyl-7-methoxydibenzo[b,d]furan-2-carbonitrile, affording 96 mg (38%) of (−)-6-sec-butyl-7-methoxydibenzo[b,d]furan-2-carbonitrile. [α]D²⁵=15° (c=0.0099 g/mL, MeOH); MS (EI) m/z 279; HPLC purity 100% at 210-370 nm, 11.6 min.; 99.5% at 244 nm, 11.6 min.; Xterra RPI8, 3.5u, 150×4.6 mm column, 1.2 mL/min, 85/15-5/95 (Ammon. Form. Buff. Ph=3.5/ACN+MeOH) for 10 min, hold 4 min.

### EXAMPLE 18

### Pharmacology

A. Effects of Progestins and Antiprogestins on Alkaline Phosphatase Activity in T47D Cells—T47D Cell Alkaline Phosphatase Assay

The purpose of this assay is to identify progestins or antiprogestins by determining a compound's effect on alkaline phosphatase activity in T47D cells. The materials and methods are as follows.

1. Reagents:

Culture medium: DMEM:F12 (1:1) (GIBCO, BRL) supplemented with 5% (v/v) charcoal stripped fetal bovine serum (not heat-inactivated), 100 U/ml penicillin, 100 ug/ml streptomycin, and 2 mM GlutaMax (GIBCO, BRL).

Alkaline phosphatase assay buffer:

I. 0.1M Tris-HCl, pH 9.8, containing 0.2% Triton X-100

II. 0.1M Tris-HCl, pH 9.8, containing 4 mM p-nitrophenyl phosphate (Sigma).

2. Cell Culture and Treatment:

Frozen T47D cells are thawed in a 37° C. water bath and diluted to 280,000 cells/ml in culture medium. To each well in a 96-well plate (Falcon, Becton Dickinson Labware), 180 μl of diluted cell suspension is added. 20 μl of reference or test compounds diluted in the culture medium is then added to each well. When testing for progestin antagonist activity, reference antiprogestins or test compounds are added in the presence of 1 nM progesterone. The cells are incubated at 37° C. in a 5% CO₂/humidified atmosphere for 24 hours. NOTE: For high throughput screening, one concentration of each compound is tested at 0.3 μg/ml. Based on an average molecular weight of 300 g/mol for the compounds in the library, the concentration is approximately 1 μM. Subsequently, active compounds will be tested in dose response assays to determine EC₅₀ and IC₅₀.

3. Alkaline Phosphatase Enzyme Assay:

At the end of treatment, the medium is removed from the plate. 50 μl of assay buffer I is added to each well. The plates are shaken in a titer plate shaker for 15 min. Then 150 μl of assay buffer II is added to each well. Optical density measurements are taken at 5 min intervals for 30 min at a test wavelength of 405 nM.

4. Analysis of Results:

Data from high throughput screen will be expressed as percent induction of alkaline phosphatase activity compared to vehicle control (agonist mode) or percent inhibition of the enzyme activity compared to 1 nM progesterone (antagonist mode). Subsequent dose response data for screening hits will be evaluated as described below. Analysis of dose-response data. For reference and test compounds, a dose response curve is generated for dose (X-axis) vs. the rate of enzyme reaction (slope) (Y-axis). Square root-transformed data are used for analysis of variance and nonlinear dose response curve fitting for both agonist and antagonist modes. Huber weighting is used to downweight the effects of outliers. EC₅₀ or IC₅₀ values are calculated from the retransformed values. JMP software (SAS Institute, Inc.) is used for both one-way analysis of variance and non-linear dose response analysis in both single dose and dose response studies.

5. Reference Compounds

Progesterone and trimegestone are reference progestins and RU486 is the reference antiprogestin. All reference compounds are run in full dose response curves and the EC₅₀ and IC₅₀ values are calculated.

Compounds that increase alkaline phosphatase activity significantly (p<0.05) compared to vehicle control are considered active. Percent (%) of induction for agonists at the tested concentrations. Antiprogestational activity is defined as including compounds that decrease 1 nM progesterone induced alkaline phosphatase activity significantly (p<0.05). EC₅₀ is the concentration of compound that gives half-maximal increase in alkaline phosphatase activity. IC₅₀ is the concentration of compound that gives half-maximal decrease in 1 nM progesterone induced phosphatase activity. The default concentration is in the nM range.

TABLE 1
Estimated EC50, standard error (SE), and 95% confidence
intervals (CI) for reference progestins from three independent experiments

| | |
|---|---|
| EC₅₀ | 95% CI |

| Compound | Exp. | (nM) | SE | lower | upper |
|---|---|---|---|---|---|
| Progesterone | 1 | 0.839 | 0.030 | 0.706 | 0.996 |
| | 2 | 0.639 | 0.006 | 0.611 | 0.669 |
| | 3 | 1.286 | 0.029 | 1.158 | 1.429 |
| Trimegestone | 1 | 0.084 | 0.002 | 0.076 | 0.091 |
| | 2 | 0.076 | 0.001 | 0.072 | 0.080 |
| | 3 | 0.160 | 0.004 | 0.141 | 0.181 |

TABLE 2
Estimated IC₅₀, standard error, and 95% confidence interval for
the reference antiprogestin RU 486 from three independent experiments

| | |
|---|---|
| EC₅₀ | 95% CI |

| Compound | Exp. | (nM) | SE | lower | upper |
|---|---|---|---|---|---|
| RU 486 | 1 | 0.103 | 0.002 | 0.092 | 0.115 |
| | 2 | 0.120 | 0.001 | 0.115 | 0.126 |
| | 3 | 0.094 | 0.007 | 0.066 | 0.134 |

B. Progesterone Receptor Whole Cell Competition Binding Assay Using T47D Cells.

This assay is to evaluate the progesterone receptor (PR) binding activity of progestins or antiprogestins in live, intact (whole) cells, using the human breast carcinoma T47D cell line and 3H -progesterone as the labeled ligand.

1. Culture medium: 5% RC: phenol red free DMEM:F12 (1:1) (GIBCO, BRL) supplemented with 5% (v/v) charcoal stripped fetal bovine serum (not heat-inactivated), 100 U/ml penicillin, 100 ug/ml streptomycin, and 2 mM GlutaMax (GIBCO, BRL). 10% RC: Same as above supplemented with 10% (v/v) FBS. ³H-Progesterone: Perkin Elmer Life Science, cat#NET-381 (typically around 102 Ci/mmol) [Liquid Scintillation Cocktail, Beckman Coulter, Ready-Safe; cat#141349, Tissue Culture Plates: 96 well, clear bottom, white, plates: VWR Part #: 29443-150 or Perkin Elmer Part #: 3983498].

2. T47D cell culture: T47D cells are maintained in 10% RC media at 37° C. in a 5% CO₂/humidified atmosphere and need to be split twice weekly for proper response. Cells are plated in 10% RC the day before binding assay at 50,000 cells per well in the white, clear bottom plates purchased through VWR or Perkin Elmer.

3. Binding Assay

Cells plated the day prior to the assay in white clear bottom plates are used. A master compound plate is set up containing control and test compounds at 20× final desired concentration for the competition binding. A typical dose range of 20× concentrations are (in nM); 200,000; 20,000; 6000; 2000; 600; 200; 20; and 2. Final concentrations are then (in nM); 10,000; 1000; 300; 100; 30; 10; 1; 0.1. Control compounds are typically run 10-fold lower than this and include a 0, or vehicle, control well. A stock of 60 nM ³H-progesterone (20×) is also prepared at a volume needed of 10 μl per well.

Media on cells is replaced with 180 μl of 5% RC. Ten microliters (10 μl) of 60 nM ³H progesterone (for final concentration of 3 nM) is added immediately followed by 10 μl of 20× test or control compounds. Compounds are incubated for 3 hrs at 37° C. (A time course study found no difference between 2 and 4 hours incubation). Following incubation, media is carefully removed and cells are washed 3× with 200 μl 5% RC each wash. 50 μl of liquid scintillation cocktail is added and the plates are shaken vigorously for a minimum of 15 minutes. Plates are read on the Wallac Microbeta 1450 plate reader.

4. Analysis of Results.

Square root-transformed data are used for analysis of variance and calculation of IC₅₀ SAS software (SAS Institute, Inc.) is used for all the statistical analysis.

5. Reference Compounds: Progesterone is used as a reference progestin and RU486 as a reference antiprogestin.

TABLE 3
Estimated IC50, standard error (SE), and 95% confidence
intervals (CI) for the reference progestin progesterone from two individual
studies.

| | |
|---|---|
| IC₅₀ | 95% CI |

| Compound | Exp. | (nM) | SE | lower | upper |
|---|---|---|---|---|---|
| Progesterone | 1 | 3.13 | 0.32 | 2.52 | 3.89 |
| | 2 | 3.58 | 0.54 | 2.61 | 4.91 |

TABLE 4
Estimated average IC50, standard error (SE), and 95%
confidence intervals (CI) for the reference antiprogestin RU486 from three
individual studies.

| | |
|---|---|
| IC₅₀ | 95% CI |

| Compound | Exp. | (nM) | SE | lower | upper |
|---|---|---|---|---|---|
| RU486 | 1 | 0.72 | 0.05 | 0.63 | 0.83 |
| | 2 | 0.50 | 0.07 | 0.37 | 0.66 |
| | 3 | 0.74 | 0.06 | 0.63 | 0.88 |

| | Identifier | | | |
|---|---|---|---|---|
| | Example 18A | Example 18A | Example 18A | |
| | ‘T47D Cell | ‘T47D Cell | ‘T47D Cell | Example 18B |
| | Alkaline | Alkaline | Alkaline | ‘T47D PR whole |
| | Phosphatase | Phosphatase | Phosphatase | cell binding |
| | Assay’ | Assay’ | Assay’ | Assay’ |
| Compound | IC₅₀ (nM) | Active Dose (nM) | Inhibition (%) | IC₅₀ (nM) |
| 7- | 259 | | | |
| methoxydibenzo | | | | |
| [b,d]furan-2- | | | | |
| carbonitrile | | | | |
| 7- | 534 | | | |
| hydroxydibenzo | | | | |
| [b,d]-furan-2- | | | | |
| carbonitrile | | | | |
| 8-fluoro-7- | | 3000 | 65 | |
| methoxy- | | | | |
| dibenzo[b,d]furan- | | | | |
| 2-carbonitrile | | | | |
| 8-fluoro-7- | | 3000 | 50 | |
| hydroxy-6-(2- | | | | |
| methylphenyl)dibenzo | | | | |
| [b,d]furan-2- | | | | |
| carbonitrile | | | | |
| 8-fluoro-6-(2- | | 3000 | 40 | |
| fluorophenyl)-7- | | | | |
| hydroxydibenzo | | | | |
| [b,d]furan-2- | | | | |
| carbonitrile | | | | |
| 6-isopropyl-7- | 48 | | | |
| methoxydibenzo | | | | |
| [b,d]-furan-2- | | | | |
| carbonitrile | | | | |
| 7-hydroxy-6- | 28 | | | 21.1 |
| isopropyldibenzo | | | | |
| [b,d]-furan-2- | | | | |
| carbonitrile | | | | |
| 6-cyclopentyl-7- | 50 | | | |
| hydroxydibenzo | | | | |
| [b,d]-furan-2- | | | | |
| carbonitrile | | | | |
| 6-sec-butyl-7- | 18.5 | | | 59.7 |
| methoxydibenzo | | | | |
| [b,d]furan-2- | | | | |
| carbonitrile | | | | |
| 6-sec-butyl-7- | 19 | | | 36.7 |
| hydroxydibenzo | | | | |
| [b,d]-furan-2- | | | | |
| carbonitrile | | | | |
| (+)-6-sec-butyl-7- | 22 | | | 41.5 |
| hydroxydibenzo | | | | |
| [b,d]furan-2- | | | | |
| carbonitrile | | | | |
| (−)-6-sec-butyl-7- | 148 | | | 81.2 |
| hydroxydibenzo | | | | |
| [b,d]furan-2- | | | | |
| carbonitrile | | | | |
| (+)-6-sec-butyl-7- | 13 | | | |
| methoxydibenzo | | | | |
| [b,d]furan-2- | | | | |
| carbonitrile | | | | |
| (−)-6-sec-butyl-7- | 30 | | | |
| methoxydibenzo | | | | |
| [b,d]furan-2- | | | | |
| carbonitrile | | | | |

All publications cited in this specification are herein incorporated by reference. While the invention has been described with reference to a particularly preferred embodiment, it will be appreciated that modifications can be made without departing from the spirit of the invention. Such modifications are intended to fall within the scope of the appended claims.

1. A compound having the structure of formula I, or a pharmaceutically acceptable salt thereof: 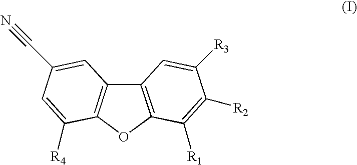
wherein R₁ is selected from the group consisting of hydrogen, halogen, alkyl, substituted alkyl, aryl, substituted aryl, heteroaryl, and substituted heteroaryl;
R₂ is selected from the group consisting of hydroxyl, alkoxy, and substituted alkoxy;
R₃ is selected from the group consisting of hydrogen and halogen; and
R₄ is selected from the group consisting of hydrogen and halogen.

2. The compound according to claim 1, wherein R₁ is selected from the group consisting of hydrogen, halogen, wherein the halogen is bromine, alkyl, wherein the alkyl is C₁-C₆ alkyl, and substituted aryl, wherein the aryl is phenyl.

3. The compound according to claim 1, wherein R₂ is selected from the group consisting of hydroxy and alkoxy, wherein the alkoxy is methoxy.

4. The compound according to claim 1, wherein R₃ is selected from hydrogen and halogen, wherein the halogen is fluorine or bromine.

5. The compound according to claim 1, wherein R₄ is selected from hydrogen and halogen, wherein the halogen is fluorine.

6. The compound according to claim 1, wherein R₁ is an alkyl, wherein the alkyl is a C₃-C₄ alkyl;
R₂ is hydroxy;
R₃ is H; and
R₄ is H.

7. The compound according to claim 1, wherein R₁ is an alkyl, wherein the alkyl is a C₃-C₄ alkyl;
R₂ is methoxy;
R₃ is H; and
R₄ is H.

8. The compound according to claim 1, wherein R₁ is an alkyl having a chiral center.

9. The compound according to claim 8, wherein the chiral center is an in the R-configuration.

10. The compound according to claim 8, wherein the compound is selected from the group consisting of:
7-methoxydibenzo[b,d]furan-2-carbonitrile;
7-hydroxydibenzo[b,d]furan-2-carbonitrile;
6-bromo-8-fluoro-7-hydroxydibenzo[b,d]furan-2-carbonitrile;
8-fluoro-7-hydroxy-6-(2-methylphenyl)dibenzo[b,d]furan-2-carbonitrile;
6-(2-chlorophenyl)-8-fluoro-7-hydroxydibenzo[b,d]furan-2-carbonitrile;
8-fluoro-7-hydroxydibenzo[b,d]furan-2-carbonitrile;
8-fluoro-7-methoxydibenzo[b,d]furan-2-carbonitrile;
8-fluoro-6-(2-fluorophenyl)-7-hydroxydibenzo[b,d]furan-2-carbonitrile;
6-isopropyl-7-methoxydibenzo[b,d]furan-2-carbonitrile;
7-hydroxy-6-isopropyldibenzo[b,a]furan-2-carbonitrile;
6-cyclopentyl-7-hydroxydibenzo[b,d]furan-2-carbonitrile;
6-sec-butyl-7-methoxydibenzo[b,d]furan-2-carbonitrile;
6-sec-butyl-7-hydroxydibenzo[b,d]furan-2-carbonitrile;
(+)-6-sec-butyl-7-hydroxydibenzo[b,d]furan-2-carbonitrile;
(−)-6-sec-butyl-7-hydroxydibenzo[b,d]furan-2-carbonitrile;
(+)-6-sec-butyl-7-methoxydibenzo[b,d]furan-2-carbonitrile;
(−)-6-sec-butyl-7-methoxydibenzo[b,d]furan-2-carbonitrile;
and salts thereof.

11. A pharmaceutical composition comprising a compound according to claim 1.

12. A method of inducing contraception, hormone replacement therapy, treating hormone-dependent disease, synchronizing estrus, treating dysmenorrhea, treating dysfunctional uterine bleeding, inducing amenorrhea, or treating symptoms of premenstrual syndrome and premenstrual dysphoric disorder in a mammal,
the method comprising administering to a mammal in need thereof a pharmaceutically effective amount of a compound of claim 1.

13. The method according to claim 12, wherein the compound is administered to induce contraception.

14. The method according to claim 12, wherein the compound is administered for hormone replacement therapy.

15. The method according to claim 12, wherein the compound is administered for treating hormone-dependent disease.

16. The method of claim 15, wherein the hormone-dependent disease is selected from the group consisting of uterine myometrial fibroids, endometriosis, benign prostatic hypertrophy; leiomyoma/fibroids, hormone dependent tumors, carcinomas and adenocarcinomas of the endometrium, colon, prostate, pituitary, and meningioma.

17. The method according to claim 16, wherein the hormone dependent cancers are selected from the group consisting of breast cancer and ovarian cancer.

18. The method according to claim 12, wherein the compound is administered for synchronizing estrus.

19. The method according to claim 12, wherein the compound is administered for treating dysmenorrhea.

20. The method according to claim 12, wherein the method is for treating dysfunctional uterine bleeding.

21. The method according to claim 12, wherein the method is for inducing amenorrhea.

22. The method according to claim 12, wherein the method is for treating symptoms of premenstrual syndrome and premenstrual dysphoric disorder in a mammal.

23. The method according to claim 12, wherein the method is for treating cycle-related symptoms.

24. The method according to claim 23, wherein said symptoms are psychological.

25. The method according to claim 24, wherein said psychological symptoms include mood changes, irritability, anxiety, lack of concentration, or decrease in sexual desire.

26. The method according to claim 23, wherein said symptoms are physical.

27. The method according to claim 26, wherein said physical symptoms include breast tenderness, bloating, fatigue, or food cravings.

28. A contraception regimen which comprises administering to a female of child bearing age for 28 consecutive days:
a) a first phase of from 14 to 24 daily dosage units of a progestational agent equal in progestational activity to about 35 to about 100 μg levonorgestrel;
b) a second phase of from 1 to 11 daily dosage units, at a daily dosage of from about 2 to 200 mg, of a compound according to claim 1; and
c) optionally, a third phase of daily dosage units of an orally and pharmaceutically acceptable placebo for the remaining days of the 28 consecutive days in which no antiprogestin, progestin or estrogen is administered; wherein the total daily dosage units of the first, second and third phases equals 28.
